(19) United States
(12) Patent Application Publication
(10) Pub. No.: US 2022/0378366 A1
(43) Pub. Date: Dec. 1, 2022

(54) BREATHING COACH APPLICATION

(71) Applicant: Kashtin COOK, Moose Jaw (CA)

(72) Inventor: Kashtin COOK, Moose Jaw (CA)

(21) Appl. No.: 17/334,891

(22) Filed: May 31, 2021

(51) Int. Cl. A61B 5/00; A61B 5/08; G16H 40/67

(52) U.S. Cl. CPC A61B 5/486; A61B 5/0816; A61B 5/7455; A61B 5/681; G16H 40/67

(57) ABSTRACT

A breathing coach system is disclosed comprising a biometric device capable of measuring the respiration rate of a user and an application monitoring the respiration rate measured by the biometric device and notifying the user when the respiration rate of the user is different from a predetermined respiration rate.

### BACKGROUND

Smart watches (or other smart biometric devices) have the ability to monitor the respiration rate of the user. This measurement is typically used to provide statistics after the fact. There is a need to use these measurements to provide instant feedback to a user to alter their respiration pattern to help with anxiety and relaxation. By taking control of their breathing in real time, the user can impact their well-being all-day long.

### BRIEF SUMMARY

In a first embodiment, a breathing coach system and method is disclosed comprising a biometric device capable of measuring the respiration rate of a user, an application coupled to the biometric device monitoring the respiration rate and notifying said user when the respiration rate of the user is different from a predetermined respiration rate.

In another embodiment, the application is executed on a processor on a smart watch.

In another embodiment, the biometric device is executed on a processor on a smart watch.

In another embodiment, the predetermined respiration rate is a baseline respiration rate recorded by said user.

In another embodiment, the predetermined respiration rate is a respiration rate following a specific pattern.

In another embodiment, the specific pattern is a 4,4,4 box breathing pattern.

In another embodiment, the notifying is done by a vibration on the smart watch.

In another embodiment, the notifying is done by playing a tone on the smart watch.

In another embodiment, the notifying is done when the monitored respiration rate is faster than the baseline respiration rate.

In another embodiment, the notifying is done to match said baseline respiration rate.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates a wearable smart device in accordance with one embodiment.

FIG. 2 illustrates an embodiment of the breathing coach system.

### DETAILED DESCRIPTION

The details of one or more embodiments of the subject matter of this specification are set forth in the accompanying drawings and the description below. Other features, aspects, and advantages of the subject matter will become apparent from the description, the drawings, and the claims.

Like reference numbers and designations in the various drawings indicate like elements.

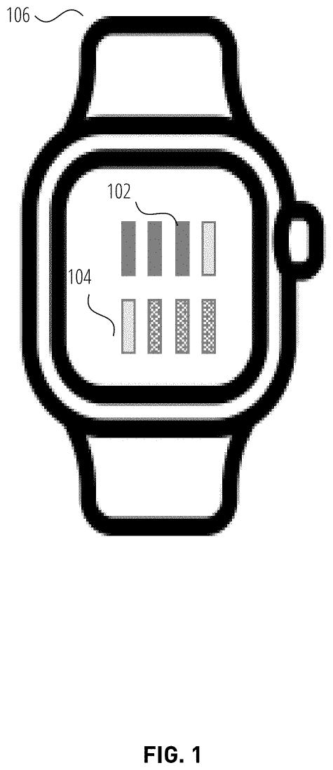

FIG. 1 depicts a wearable smart device 106 (for example, but not limited to a smart watch) capable of measuring respiration rate or receiving respiration rate measurements from a biometric device. An application downloaded on the wearable smart device 106 displays bars reflecting the inhale level 102 and the exhale level 104 of the users as measured and compared to a baseline natural conscious breathing of the user.

In another embodiment, the biometric device is a separate device coupled with the application device via a communication protocol.

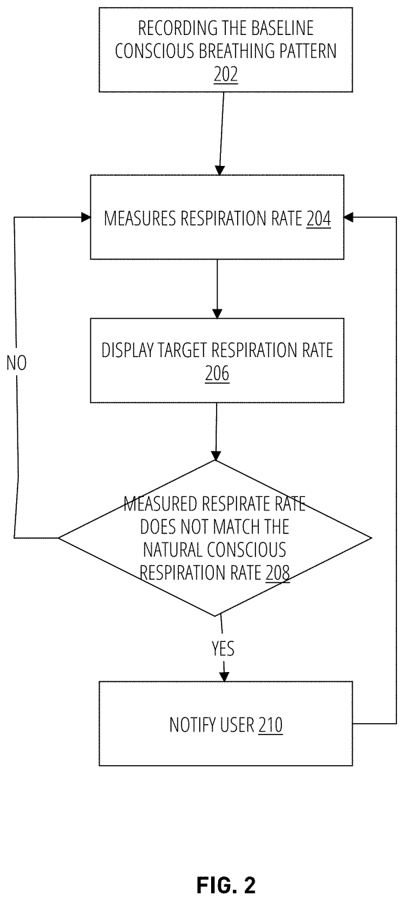

As per FIG. 2, the user initializes the application by recording the baseline conscious breathing pattern 202. The conscious breathing pattern is stored in the wearable smart device 106 as the baseline.

Then the application measures respiration rate 204 and displays target respiration rate 206 by showing the inhale level 102 and exhale level 104 on the screen of the wearable smart device 106. The levels and speed can be shown using bars as shown in FIG. 1. For example, the inhale level 102 is shown with bars lighting up from left to right and the exhale level 104 with the bars going from right to left. If the application is configured to have a pause between the inhale and the exhale, and/or the exhale and the inhale, then another graphic icon can be used as a counter for the pause.

The display could be of any designs that allow to show the two dimension of the respiration, the rate and the depth.

If the measured respirate rate does not match the baseline 208, the user is notified 210 using a vibration or a sound generated by the wearable smart device 106. For example if the user tries to exhale before the 4th bar of the inhale is reached.

The user can configure the application to make different sounds, types of vibrations and levels thereof.

The application measures respiration rates 204 on an ongoing basis and continuously compares to the baseline to provide continuous feedback. Optionally, the application can be configured via setting to only provide feedback during specific times of day.

As the user goes on about its day and forgets to breath consciously, the watch will constantly remind the user to go back to their baseline natural conscious breathing pattern.

In another embodiment, the application can be configured to implement different types of breathing rhythms including a pause after the inhale and/or before the exhale. Optionally, the application can use different types of vibration for the inhale, pause, exhale. For example, but not limited to, the 4,4,4 box breathing technic used navy seals. Any other types of breathing techniques can be programmed into the application.

Using the application, the user does not need to worry about counting and they can focus on the breath and visualize toxic energy leaving their body. A subtle vibration of the wearable smart device 106 on the wrist triggers the user to inhale and hold their breath for 4 seconds, then another vibration will notify the user to exhale. The application acts as a metronome for the breathing of the user causing ongoing real-time relaxation.

In another embodiment, the application on the wearable smart device 106 will cause the user to take deeper breaths than the baseline to increase relaxation. Using this setting the vibration may optionally feel different or a different sound can be played. In this embodiment, the application acts as a metronome for breathing.

In another embodiment, the application records the breathing pattern of the user. Using this data, the application can display the breathing pattern in comparison with previous time segments or in comparison with the baseline conscious breathing pattern to show improvements achieved using the notifications.

In another embodiment, the application provides statistics of the number of time the notifications are sent, these statistics can show the user improvement over time.

The foregoing descriptions of specific embodiments of the present invention have been presented for purposes of illustration and description. They are not intended to be exhaustive or to limit the invention and method of use to the precise forms disclosed. Obviously, many modifications and variations are possible in light of the above teaching. The embodiments described were chosen and described in order to best explain the principles of the invention and its practical application, and to thereby enable others skilled in the art to best utilize the invention and various embodiments with various modifications as are suited to the particular use contemplated. It is understood that various omissions or substitutions of equivalents are contemplated as circumstance may suggest or render expedient but is intended to cover the application or implementation without departing from the spirit or scope of the claims of the present invention.

What is claimed is:

1. A breathing coach system comprising:
a biometric device capable of measuring the respiration rate of a user;
an application monitoring the respiration rate on a smart device coupled to the biometric device and notifying said user when the respiration rate of the user is different from a predetermined respiration rate.

2. The system of claim 1 wherein said application is executed on a processor on a smart device.

3. The system of claim 1 wherein said biometric device is executed on a processor on a smart device.

4. The system of claim 1 where said predetermined respiration rate is a baseline respiration rate recorded by said user.

5. The system of claim 1 where said predetermined respiration rate is a respiration rate following a specific pattern.

6. The system of claim 5 where said specific pattern is a 4,4,4 box breathing pattern.

7. The system of claim 1 wherein said notifying is done by a vibration on the smart device.

8. The system of claim 1 wherein said notifying is done by playing a tone on the smart device.

9. The system of claim 1 wherein said notifying is done when the monitored respiration rate is faster than the baseline respiration rate.

10. The system of claim 1 wherein said notifying is done to match said baseline respiration rate.

11. A method for coaching a user's breathing rate comprising:
measuring a respiration rate of a user using a biometric device;
monitoring a respiration rate of the user using an application on a smart device coupled to said biometric device and notifying said user when the respiration rate of the user is different from a predetermined respiration rate.

12. The method of claim 11 wherein said application is executed on a processor on a smart device.

13. The method of claim 11 wherein said biometric device is executed on a processor on a smart device.

14. The method of claim 11 where said predetermined respiration rate is a baseline respiration rate recorded by said user.

15. The method of claim 11 where said predetermined respiration rate is a respiration rate following a specific pattern.

16. The method of claim 15 where said specific pattern is a 4,4,4 box breathing pattern.

17. The method of claim 11 wherein said notifying is done by a vibration on the smart device.

18. The method of claim 11 wherein said notifying is done by playing a tone on the smart device.

19. The method of claim 11 wherein said notifying is done when the monitored respiration rate is faster than the baseline respiration rate.

20. The method of claim 11 wherein said notifying is done to match said baseline respiration rate.
